SEQUENCE LISTING (18 sequences)

SEQ ID NO: 1 - RNA, 19 nt, Artificial Sequence
  Sense strand of human CD40 siRNA
ugccuuccuugcggugaaa

SEQ ID NO: 2 - RNA, 19 nt, Artificial Sequence
  Antisense strand of human CD40 siRNA
uuucaccgcaaggaaggca

SEQ ID NO: 3 - RNA, 19 nt, Artificial Sequence
  Sense strand of human CD40 siRNA
gcgaauuccuagacaccug

SEQ ID NO: 4 - RNA, 19 nt, Artificial Sequence
  Antisense strand of human CD40 siRNA
caggugucuaggaauucgc

SEQ ID NO: 5 - RNA, 19 nt, Artificial Sequence
  Sense strand of human CD40 siRNA
ugucacccuuggacaagcu

SEQ ID NO: 6 - RNA, 19 nt, Artificial Sequence
  Antisense strand of human CD40 siRNA
ugcuuguccaagggugaca

SEQ ID NO: 7 - RNA, 19 nt, Artificial Sequence
  Sense strand of human CD40 siRNA
uuuucccgacgaucuuccu

SEQ ID NO: 8 - RNA, 19 nt, Artificial Sequence
  Antisense strand of human CD40 siRNA
aggaagaucgucgggaaaa

SEQ ID NO: 9 - RNA, 19 nt, Artificial Sequence
  Sense strand of human CD40 siRNA
ccacccacugcaugcagag

SEQ ID NO: 10 - RNA, 19 nt, Artificial Sequence
  Antisense strand of human CD40 siRNA
cucugcaugcagugggugg

SEQ ID NO: 11 - RNA, 19 nt, Artificial Sequence
  Sense strand of human CD40 siRNA
cuggugagugacugcacag

SEQ ID NO: 12 - RNA, 19 nt, Artificial Sequence
  Antisense strand of human CD40 siRNA
cugugcagucacucaccag

SEQ ID NO: 13 - RNA, 19 nt, Artificial Sequence
  Sense strand of human CD40 siRNA
cagagagacacacugccac

SEQ ID NO: 14 - RNA, 19 nt, Artificial Sequence
  Antisense strand of human CD40 siRNA
guggcagugugucucucug

SEQ ID NO: 15 - RNA, 19 nt, Artificial Sequence
  Sense strand of human CD40 siRNA
uacugcgaccccaaccuag

SEQ ID NO: 16 - RNA, 20 nt, Artificial Sequence
  Antisense strand of human CD40 siRNA
cuagguuggggucgccagua

SEQ ID NO: 17 - RNA, 27 nt, Artificial Sequence
  Sense strand of human CD40 siRNA
gaaagcgaauuccuagacaccuggaac

SEQ ID NO: 18 - RNA, 27 nt, Artificial Sequence
  Antisense strand of human CD40 siRNA
guuccaggugucuaggaauucgcuuuc

(19) United States
(12) Patent Application Publication
(10) Pub. No.: US 2014/0315981 A1
(43) Pub. Date: Oct. 23, 2014

(54) STRATEGIES FOR PREVENTION AND/OR TREATMENT OF DISEASES BASED ON CD40 SILENCING

(71) Applicants: Josep Maria Grinyo i Boira, Alella (ES); Oriol Bestard Matamoros, Barcelona (ES); Josep Maria Gruzado Garrit, Castelldefels (ES); Juan Torres Ambros, Barcelona (ES); Josep M. Aran Perramon, Mollet del Valles (ES)

(72) Inventors: Josep Maria Grinyo i Boira, Alella (ES); Oriol Bestard Matamoros, Barcelona (ES); Josep Maria Gruzado Garrit, Castelldefels (ES); Juan Torres Ambros, Barcelona (ES); Josep M. Aran Perramon, Mollet del Valles (ES)

(73) Assignee: FUNDACIO INSTITUT D'INVESTIGACIO BIOMEDICA DE BELLVITGE (IDIBELL), Hospitalet de Llobregat (ES)

(21) Appl. No.: 14/343,558

(22) PCT Filed: Sep. 7, 2012
(86) PCT No.: PCT/EP2012/067503

§ 371 Date: May 1, 2014

(30) Foreign Application Priority Data
Sep. 7, 2011 (ES) 11382286.0

(51) Int. Cl. C12N 15/113

(52) U.S. Cl. CPC C12N 15/1138
USPC 514/44 A

(57) ABSTRACT

The present invention is related to methods for prevention and/or treatment of a number of diseases, such as lupus nephritis, ischemia/reperfusion injury and sepsis, based on the silencing of CD40 using different RNA silencing strategies.

### TECHNICAL FIELD

The present invention relates to the field of immunomodulation and, more in particular, to methods for prevention and/or treatment of a number of diseases based on the silencing of CD40 using RNA interference strategies.

### BACKGROUND ART

### Lupus Nephritis

The incidence of end stage renal disease attributed to systemic lupus erythematosus (SLE) continues to rise despite the availability of potent therapies, which apparently provide reno-protection. Some overt clinical evidence of renal involvement at the time of diagnosis is expected in about two-thirds of patients with well documented SLE, and renal disease remains a leading cause of morbidity and mortality in the short term. Available data indicate that cyclophosphime (CYP) and steroids can effectively delay the progression of renal disease in lupus nephritis. Nevertheless, failure to achieve remission is reported in 18-57% of patients receiving CYP and toxicity-related complications limit its use in the long term. Mycophenolate mofetil has subsequently been shown to have a role among treatment options for lupus nephritis (Corna D et al. Kidney Int 1997; 51: 1583-1589, Chan T M et al. N Engl J Med 2000; 343:1156-1162) although the available data have yet to confirm that its efficacy is comparable to that of CYP combined with steroids (Flanc R S et al. Am J Kidney Dis 2004; 43: 197-208). Female NZB/W F1 hybrid mice spontaneously develop an autoimmune disease which resembles human SLE and whose main feature is the formation of autoantibodies against multiple epitopes of chromatin. Nucleosomes (DNA complexed to histones) are known to be generated by apoptosis. When there is insufficient removal of apoptotic cells, as in SLE, nucleosomes act as autoantigens and drive a T-cell immune response, leading to the formation of autoantibodies, which bind to the glomerular basement membrane and promote inflammation.

Costimulatory signals are involved in the pathogenesis of SLE. In this regard, besides the classical use of xenobiotics in murine models of SLE (Alperovich G et at Lupus 2007; 16:18-24), blocking costimulatory molecules interactions may also be promising. The interference of CD40-CD40 ligand interaction with mAbs and the CD28-B7 interaction with a soluble cytotoxic T-lymphocyte antigen 4 (CTLA-4)-IgG1 construct (Abatacept), have also been attempted as a therapeutic strategy for SLE (Gaged M & Gordon C Curr Opin Investig Drugs. 2010; 11:1256-64)

### Ischemia/Reperfusion Injury

Renal dysfunction appearing within the first 7 to 10 days post-transplantation is known as Delayed Graft Function (DGF), it often requires temporizing hemodialysis, and may be a harbinger of future allograft rejection or dysfunction. Analysis of more than 138,000 cases of renal transplantation from the UNOS Renal Transplant Registry database has shown that long term graft survival (>10 years) has remained unchanged despite improvements in short term acute rejection rates. One of the major contributing factors to poorer long-term outcomes identified in this retrospective review was DGF. The intimate source of tissue injury leading to DGF is poorly understood, although ischemia/reperfusion-injury has been clearly identified as an antigen-independent risk factor in animal models.

Although anti-CD40L antibodies have been shown to be effective in preventing acute rejection and induce tolerance in some transplantation models much less is known regarding the potential value of interfering with the CD40-CD40L signal in ischemia/reperfusion-injury (Yamada A & Sayegh M H Transplantation 2002; 73: S36-S39). The importance of CD40L in hepatic ‘warm’ ischemia/reperfusion-injury has been well documented in a non-transplant murine knockout model (Shen X D, et al, Transplantation 2002; 74:315-319) as well as in a more clinically relevant rat model of ex vivo “cold” ischemia followed by orthotopic liver transplantation (Ke B et al, Mol Ther. 2004; 9: 38-45). More recent work from the same group suggests that CD4 T-lymphocytes function in liver ischemia/reperfusion-injury via CD40L without de novo antigen-specific activation, and that innate immunity induces CD40 up-regulation with the consequent facilitation of CD40-CD40L signaling to induce tissue injury (Shen X et al, Hepatology 2009; 50:1537-46).

### Sepsis

Sepsis is a systemic response to infection, and septic shock develops in a number of patients after surgery as a complication. Sepsis is the leading cause of death in critically ill patients, and the incidence of sepsis is increasing. The mortality rate of severe sepsis is very high (up to 70%), and the calculated costs exceed $15 billion per year in the United States. The rate of severe sepsis during hospitalization almost doubled during the last decade and is considerably greater than previously predicted. Sepsis causes multiorgan failure, including acute kidney injury (AKI), and patients with both sepsis and AKI have an especially high mortality rate. A multinational prospective observational study including 29,269 critically ill patients revealed that the occurrence of AKI in the intensive care unit was approximately 6%, the most frequent contributing factor to AKI being sepsis (50%). Other reports showed that between 45% and 70% of all AKI is associated with sepsis.

The most common cause of sepsis is exposure to the structural component of a Gram-negative bacteria membrane, LPS, and key symptoms include hypotension and vasoplegia, which may lead to the multiple organ dysfunction and ultimately death.

The mechanism of LPS toxicity requires the active response of host cells (Rietschel E T et at FASEB J 1994; 8:217-225). LPS, through its lipid A component, interacts with various host cell types including mononuclear cells, endothelial and smooth muscle cells, polymorphonuclear granulocytes, and thrombocytes, among which macrophages/monocytes are of particular importance. Thus, LPS-induced activation of macrophages results in the production of bioactive lipids, reactive oxygen species, and in particular, peptide mediators such as tumor necrosis factor a (TNF), interleukin 1 (IL-1), IL-6, IL-8, and IL-10. These secondary, hormone-like proteins are endowed with potent bioactivities and are capable of inducing many of the typical endotoxin effects by acting independently, in sequence, synergistically or antagonistically. It appears that beneficial effects (e.g., induction of resistance to infection, adjuvant activity) are elicited when low levels of mediators are produced and that detrimental effects (e.g., high fever, hypotension, irreversible shock) are induced when high levels of mediators reach the circulation. However, low mediator concentrations may also become harmful when the host organism is in a hyperreactive state to LPS. Hyperreactivity to endotoxin may be caused by exotoxins, chronic infection, and by growing tumors, and one important factor contributing to sensitization to LPS has been identified as γ-interferon.

Continuing concern over the efficacy and safety of the only FDA-approved therapy for severe sepsis (activated protein C) highlights the critical need to improve our understanding of the pathophysiology of sepsis and to develop novel treatment strategies for critically ill patients (Riedemann N C et al. J Clin Invest 2003; 112:460-467).

The interference of the interaction CD40-CD40L as therapeutic strategy against sepsis has been attempted by e.g. Schwulst et al., which discloses monoclonal antibodies against CD40 to protect lymphocytes from sepsis-induced apoptosis (Schwulst S J et al. J Immunol 2006; 177:557-565). However, anti-CD40L monoclonal antibodies have been shown to result in a high rate of thromboembolic complications derived from the activation and aggregation of platelets, which express CD40L (Kawai T et al. Nat. Med. 2000; 6: 114).

Therefore, it is necessary to develop alternative strategies for disrupting CD40 signaling capable of preventing sepsis and that overcome the problems associated to the methods based on the use of anti-CD40 antibodies.

### BRIEF SUMMARY OF THE INVENTION

The invention relates to interfering RNAs that silence CD40 gene expression or polynucleotides coding said interfering RNAs for their use in the prevention and/or the treatment of a disease in a subject, wherein said disease is selected from the group consisting of: sepsis, lupus nephritis, and renal ischemia/reperfusion injury.

### BRIEF DESCRIPTION OF THE FIGURES

FIG. 1 shows the survival of NZB/W mice in percentage among the groups with drug therapy (cyclophosphime CYP, CTL4, siRNA 1 dose/week and siRNA 2 doses/week). Not significant difference was found.

FIG. 2 shows the anti-DNA titers in the groups of NZB/W mice (control, CYP, CTL4, siRNA 1 dose/week and siRNA 2 doses/week) are shown.

FIG. 3 shows the proteinuria levels in the three groups of NZB/W mice (control, CYP, CTL4 and siRNA).

FIG. 4 shows the ratio of proteinuria levels (mg) and creatinine levels (mg) in the groups analyzed (control, CYP, CTL4, siRNA 1 dose/week and siRNA 2 doses/week)

FIG. 5 is a representation of the main histological findings assessed for the analyzed groups (control, CYP, CTL4, siRNA once per week and siRNA twice per week) in a semi-quantitative scale.

FIG. 6 represents the glomerular IgG deposition for the analyzed groups (control, CYP, CTLA4 and siRNA).

FIG. 7 represents the C3 glomerular deposition for the analyzed groups (control, CYP, CTLA4 and siRNA).

FIG. 8 is a representation of the spleen weight in the analyzed groups (control, CYP, CTLA4 and siRNA).

FIG. 9 shows the percentage of CD19⁺, CD19⁺ CD25⁺, CD19⁺ CD69⁺ and CD19⁺ CD25⁺ CD69⁺ cells in the splenocytes for the analyzed groups. (CTLA4, CYP, siRNA once per week and siRNA twice per week).

FIG. 10 shows the number of intra-renal CD3 T+ cells in NZB/w mice administered CTLA4, CYP, CD40 siRNA once a week and siRNA twice a week.

FIG. 11 shows the fluorescence of renal tissue after intravenous injection of CD40 siRNA labeled with Cy 5.5.

FIG. 12 shows localization of CD40 siRNA in renal tubules after intravenous injection of CD40 siRNA labeled with Cy 5.5.

FIG. 13 shows the expression of CD40 in the kidney and the liver of different groups of mice after LPS injection. Mice were injected intraperitoneally (IP) or intravenously (IV) Cy-5.5 fluorescent CD40 siRNA.

### DETAILED DESCRIPTION OF THE INVENTION

Use of Interfering siRNA Specific for CD40 for the Treatment of Lupus Nephritis

The authors of the present invention have found that the silencing of CD40 gene expression by RNA interference results in the attenuation of the histological lesions and proteinuria in a mouse model of lupus nephritis to similar levels as the gold-standard therapy using cyclophosphamide (see example 1).

Thus, in a first aspect, the invention relates to an interfering RNA which silences CD40 gene expression or a polynucleotide coding for said interfering RNA for use in the prevention and or the treatment of lupus nephritis.

Alternatively, the invention relates to the use of an interfering RNA that silences CD40 gene expression or a polynucleotide coding for said interfering RNA for the manufacture of a medicament for the treatment of lupus nephritis.

Alternatively, the invention relates to a method for the prevention and/or the treatment of lupus nephritis in a subject in need thereof which comprises the administration to said subject of an interfering RNA that silences CD40 gene expression or a polynucleotide coding for said interfering RNA.

The term “prevention” is understood to mean the administration of an oligonucleotide according to the invention or of a medicament containing it in an initial or early stage of the disease, or also to avoid its appearance.

As used herein, the term “treatment” refers to both therapeutic measures and prophylactic or preventative measures, wherein the object is to prevent or slow down (lessen) an undesired physiological change or disorder, such as the acute rejection after a renal transplant. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, cattle, cows, and so on. In a preferred embodiment of the invention, the subject is a mammal. In a more preferred embodiment of the invention, the subject is a human.

The term “lupus nephritis”, as used herein, refers to an inflammation of the kidney caused by systemic lupus erythematosus (SLE), a disease of the immune system. SLE typically causes harm to the skin, joints, kidneys, and brain. Lupus nephritis may cause weight gain, high blood pressure, dark urine, or swelling around the eyes, legs, ankles, or fingers.

Lupus nephritis (LN) occurs in more than one-third of patients with systemic lupus erythematosus. Its pathogenesis is mostly attributable to the glomerular deposition of immune complexes and overproduction of T helper-(Th-) 1 cytokines. In this context, the high glomerular expression of IL-12 and IL-18 exerts a major pathogenetic role. These cytokines are locally produced by both macrophages and dendritic cells which attract other inflammatory cells leading to maintenance of the kidney inflammation. However, other populations including T-cells and B-cells are integral for the development and worsening of renal damage. T-cells include many pathogenetic subsets, and the activation of Th-17 in keeping with defective T-regulatory (Treg) cell function regards as further event contributing to the glomerular damage. These populations also activate B-cells to produce nephritogenic auto-antibodies (Tucci M et at J Biomed Biotechnol 2010; 2010:1-6).

The expression “an interfering RNA that silences CD40 gene expression”, as used herein, relates to a RNA molecule which is capable of causing degradation of CD40 the mRNA and an inhibition of translation by the process of RNA interference. Suitable means for determining whether a given interfering RNA is capable of silencing CD40 include any means for determining the levels of the CD40 mRNA in a sample, including RT-PCR, Northern blot and the like as well as any means for determining the levels of CD40 protein, including immunological methods such as ELISA, Western blot, immunohistochemistry. An interfering RNA is considered as capable of silencing CD40 when cells treated with the interfering RNA or which express the interfering RNA as a consequence of having been contacted with a polynucleotide encoding said interfering RNA when it results in a decrease of at least 10%, at least 20%, at least 30%, at least 40%, at least 50%, at least 60%, at least 70%, at least 80%, at least 90% or 100% in the levels of the CD40 mRNA or CD40 protein with respect to the same cells which have not been contacted with the interfering RNA or the polynucleotide encoding said interfering RNA.

The expression “RNA interference” or RNAi is a process of sequence-specific post-transcriptional gene repression which can occur in eukaryotic cells. In general, this process involves degradation of an mRNA of a particular sequence induced by double-stranded RNA (dsRNA) that is homologous to that sequence. This dsRNA is capable of causing the silencing of gene expression by means of converting said RNA into siRNA by means of an RNase type III (Dicer). One of the siRNA strands is incorporated into the ribonucleoprotein complex referred to as the RNA-induced silencing complex (RISC). The RISC complex uses this single strand of RNA to identify mRNA molecules that are at least partially complementary to the RNA strand of the siRNA incorporated in the RISC that are degraded or undergo an inhibition in their translation. Thus, the siRNA strand that is incorporated into the RISC is known as a guide strand or antisense strand. The other strand, which is known as a transient strand or sense strand, is eliminated from the siRNA and is partly homologous to the target mRNA. The degradation of a target mRNA by means of the RISC complex results in a reduction in the expression levels of said mRNA and of the corresponding protein encoded thereby. Furthermore, RISC can also cause the reduction in the expression by means of the inhibition of the translation of the target mRNA.

The invention contemplates the use of interfering RNA specific for CD40 as such as well as the use of polynucleotides encoding for said interfering RNA.

As used herein, the term “specific for CD40” refers to small inhibitory RNA duplexes that, by means of showing a substantial degree of sequence complementarity with CD40 mRNA, induce the RNA interference (RNAi) pathway to negatively regulate gene expression of CD40.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50 degrees centigrade or 70 degrees centigrade for 12-16 hours followed by washing. Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides.

Specific interfering RNAs include RNAs that show base-pairing to the target polynucleotide over the entire length of the first and second nucleotide sequence. Such sequences can be referred to as “fully complementary” with respect to each other herein. However, where a first sequence is referred to as “substantially complementary” with respect to a second sequence herein, the two sequences can be fully complementary, or they may form one or more, but generally not more than 4, 3 or 2 mismatched base pairs upon hybridization, while retaining the ability to hybridize under the conditions most relevant to their ultimate application. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, may yet be referred to as “fully complementary.”

“Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs includes, but not limited to, G:U Wobble or Hoogstein base pairing.

The terms “complementary”, “fully complementary” and “substantially complementary” herein may be used with respect to the base matching between the sense strand and the antisense strand of a dsRNA, or between the antisense strand of a dsRNA and a target sequence, as will be understood from the context of their use.

As used herein, a polynucleotide which is “substantially complementary to at least part of a messenger RNA (mRNA) refers to a polynucleotide which is substantially complementary to a contiguous portion of the mRNA of interest (e.g., encoding target gene). For example, a polynucleotide is complementary to at least a part of a target gene mRNA if the sequence is substantially complementary to a non-interrupted portion of a mRNA encoding target gene. As used herein the term “oligonucleotide” embraces both single and double stranded polynucleotides.

The double stranded oligonucleotides used to effect RNAi are preferably less than 50 base pairs in length and, more preferably, comprise about 25, 24, 23, 22, 21, 20, 19, 18 or 17 base pairs of ribonucleic acid. Optionally the dsRNA oligonucleotides of the invention may include 3′ overhang ends. Exemplary 2-nucleotide 3′ overhangs may be composed of ribonucleotide residues of any type and may even be composed of 2′-deoxythymidine residues, which lowers the cost of RNA synthesis and may enhance nuclease resistance of siRNAs in the cell culture medium and within transfected cells (see Elbashir et al., Nature 411: 494-8, 2001). Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discernable to the skilled artisan. Exemplary dsRNAs may be synthesized chemically or produced in vitro or in vivo using appropriate expression vectors. Exemplary synthetic RNAs include 21 nucleotide RNAs chemically synthesized using methods known in the art (e.g., Expedite RNA phosphoramidites and thymidine phosphoramidite (Proligo, Germany). Synthetic oligonucleotides are preferably deprotected and gel-purified using methods known in the art (see, e.g., Elbashir et al., Genes Dev. 15: 188-200, 2001). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence. Any of the above RNA species will be designed to include a portion of nucleic acid sequence represented in a target nucleic acid, such as, for example, a nucleic acid that hybridizes, under stringent and/or physiological conditions, to the polynucleotide encoding human CD40.

The specific sequence utilized in design of the interfering RNA for use according to the present invention may be any contiguous sequence of nucleotides contained within the expressed CD40 gene message. Programs and algorithms, known in the art, may be used to select appropriate target sequences. In addition, optimal sequences may be selected utilizing programs designed to predict the secondary structure of a specified single stranded nucleic acid sequence and allowing selection of those sequences likely to occur in exposed single stranded regions of a folded mRNA. Methods and compositions for designing appropriate oligonucleotides may be found, for example, in U.S. Pat. No. 6,251,588, Birmingham, A. et al. 2007, Nature Protocols, 2:2068-2078, Ladunga, I. 2006, Nucleic Acids Res. 35:433-440 and Martineau, H., Pyrah, I., 2007, Toxicol. Pathol., 35:327-336 and Pei and Tuschl, 2006, Nature Methods, 3:670-676, the contents of which are incorporated herein by reference. Messenger RNA (mRNA) is generally thought of as a linear molecule which contains the information for directing protein synthesis within the sequence of ribonucleotides, however studies have revealed a number of secondary and tertiary structures that exist in most mRNAs. Secondary structure elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three dimensional structure. A number of researchers have measured the binding energies of a large number of RNA duplex structures and have derived a set of rules which can be used to predict the secondary structure of RNA (see, e.g., Jaeger et al., Proc. Natl. Acad. Sci. USA 86: 7706, 1989; and Turner et al., Annu Rev. Biophys. Biophys. Chem. 17:167, 1988). The rules are useful in identification of RNA structural elements and, in particular, for identifying single stranded RNA regions which may represent preferred segments of the mRNA to target for silencing RNAi, ribozyme or antisense technologies. Accordingly, preferred segments of the mRNA target can be identified for design of the RNAi mediating dsRNA oligonucleotides as well as for design of appropriate ribozyme and hammerhead ribozyme compositions of the invention.

Different types of molecules, such as small interfering RNA and short hairpin RNA, have been used effectively in the RNAi technology.

Small interfering RNA (siRNA), sometimes known as short interfering RNA or silencing RNA, are a class of double-stranded RNA molecules that play a variety of roles in biology. Most notably, siRNA is involved in the RNA interference (RNAi) pathway where the siRNA interferes with the expression of a specific gene. In addition to their role in the RNAi pathway, siRNAs also act in RNAi-related pathways, e.g., as an antiviral mechanism or in shaping the chromatin structure of a genome. Synthetic siRNAs have been shown to be able to induce RNAi in mammalian cells. This discovery led to a surge in the use of siRNA/RNAi for biomedical research and drug development.

A siRNA can be chemically synthesised or can be obtained through in vitro transcription. siRNAs typically consist of a double RNA strand with a length between 15 and 40 nucleotides and can contain a 3′ and/or 5′ overhanging region with 1 to 6 nucleotides. As used herein, a “nucleotide overhang” refers to the unpaired nucleotide or nucleotides that protrude from the duplex structure of a siRNA when a 3′-end of one strand of the siRNA extends beyond the 5′-end of the other strand, or vice versa. “Blunt” or “blunt end” means that there are no unpaired nucleotides at that end of the siRNA, i.e., no nucleotide overhang. A “blunt ended” siRNA is a siRNA that is double-stranded over its entire length, i.e., no nucleotide overhang at either end of the molecule. The length of the overhanging region is independent of the total length of the siRNA molecule. siRNAs act by means of the degradation or the post-transcriptional silencing of the target messenger. The siRNAs of the invention are substantially homologous with a pre-selected region of the target CD40 mRNA. “Substantially homologous” is understood as that they have a sequence which is sufficiently complementary or similar to the target mRNA such that the siRNA is capable of causing the degradation thereof by RNA interference. The siRNAs suitable for causing said interference include siRNAs formed by RNA, as well as siRNAs containing different chemical modifications such as:
siRNAs in which the bonds between the nucleotides are different from those that occur in nature, such as phosphorothioate bonds,
conjugates of the siRNA strand with a moiety that promotes penetration of the siRNA into biological membranes. In a particular example, the siRNA may be modified by coupling to a cholesterol molecule. The cholesterol conjugate may be coupled to the 5′ or to the 3′ end of the siRNA with a functional reagent, such as a fluorophore,
modifications of the ends of the siRNA strands, particularly the 3′ end by means of the modification with different functional groups of the hydroxyl in position 2′,
nucleotides with modified sugars such as O-alkylated moieties in position 2′ such as 2′-O-methylribose p 2′-O-fluororibose,
nucleotides with modified bases like halogenated bases (for example 5-bromouracil and 5-iodouracil), alkylated bases (for example 7-methylguanosine).

In a preferred embodiment, the siRNAs for use according to the present invention comprise two overhanging nucleotides at the 3′ end of each of the RNA strands, are stabilized with a partial phosphorothioate backbone, contains 2′-O-methyl sugar modification on the sense and antisense strands and additionally has a cholesterol conjugate to the 3′ end of the sense strand by means of a pyrrolidine linker.

The siRNAs of the invention can be obtained using a series of techniques well-known to a person skilled in the art. For example, the siRNA can be chemically synthesised starting from ribonucleosides protected with phosphoramidite groups in a conventional DNA/RNA synthesizer.

Short hairpin RNA (shRNA) is yet another type of RNA that may be used to effect RNAi. An shRNA is a RNA molecule formed by two anti parallel strands connected by a hairpin region and wherein the sequence of one of the anti parallel strands is complementary to a pre-selected region in the target mRNA. The shRNAs are formed by a short antisense sequence (with 19 to 25 nucleotides), followed by a loop of 5 to 9 nucleotides followed by the sense strand. shRNAs can be chemically synthesized from ribonucleosides protected with phosphoramidite groups in a conventional DNA/RNA synthesizer or they can be obtained from a polynucleotide by means of in vitro transcription. shRNAs are processed inside the cell by the RNase Dicer that eliminates the hairpin region giving rise to siRNAs as has been previously described. shRNAs can also contain distinct chemical modifications as has been previously described in the case of siRNAs.

Currently, short-interfering RNAs (siRNAs) and short-hairpin RNAs (shRNAs) are being extensively used to silence various genes to silence functions carried out by the genes. It is becoming easier to harness RNAi to silence specific genes, owing to the development of libraries of ready-made shRNA and siRNA gene-silencing constructs by using a variety of sources. For example, RNAi Codex, which consists of a database of shRNA related information and an associated website, has been developed as a portal for publicly available shRNA resources and is accessible at http://codex.cshl.org. RNAi Codex currently holds data from the Hannon-Elledge shRNA library and allows the use of biologist-friendly gene names to access information on shRNA constructs that can silence the gene of interest. It is designed to hold user-contributed annotations and publications for each construct, as and when such data become available. Olson et al. (Nucleic Acids Res. 34(Database issue): D153-D157, 2006, incorporated by reference) have provided detailed descriptions about features of RNAi Codex, and have explained the use of the tool. All these information may be used to help design the various siRNA or shRNA targeting AMPA receptor or other proteins of interest.

In another aspect, the invention contemplates the use of a polynucleotide which encodes for the interfering RNA specific for CD40.

A “polynucleotide”, “nucleic acid,” or “nucleic acid molecule” is a polymeric compound comprised of covalently linked subunits called nucleotides. Nucleic acid includes polyribonucleic acid (RNA) and polydeoxyribonucleic acid (DNA), both of which may be single-stranded or double-stranded. DNA includes cDNA, genomic DNA, synthetic DNA, and semi-synthetic DNA.

The “polynucleotide coding for an interfering RNA that silences CD40 gene expression” is a polynucleotide the transcription of which gives rise to the previously described siRNA or shRNA. This polynucleotide comprises a single promoter region regulating the transcription of a sequence comprising the sense and antisense strands of the shRNAs and miRNAs connected by a hairpin or by a stem-loop region. In principle, any promoter can be used for the expression of the shRNAs and miRNAs provided that said promoters are compatible with the cells in which the siRNAs are to be expressed. Thus, the promoters suitable for carrying out this invention include those for the expression of genes whose expression is specific of renal cells. Gene promoters specific of renal cells include, but are not limited to, the uromodulin promoter, the Tamm-Horsfall protein promoter or the type 1 gamma-glutamyltranspeptidase promoter.

In addition, the polynucleotides encoding siRNAs may comprise two transcriptional units, each formed by a promoter regulating the transcription of one of the strands formed in siRNA (sense and antisense). The polynucleotides encoding siRNAs can contain convergent or divergent transcriptional units. In the divergent transcription polynucleotides, the transcriptional units encoding each of the DNA strands forming the siRNA are located in tandem in the polynucleotide such that the transcription of each DNA strand depends on its own promoter, which can be the same or different (Wang, J. et al., 2003, Proc. Natl. Acad. Sci. USA, 100:5103-5106 and Lee, N. S., et al., 2002, Nat. Biotechnol., 20:500-505). In the convergent transcription polynucleotides, the DNA regions giving rise to the siRNAs form the sense and antisense strands of a DNA region that is flanked by two inverted promoters. After the transcription of the sense and antisense RNA strands, they will form the hybrid corresponding to the functional siRNA.

The polynucleotides encoding for the siRNAs or for the shRNAs of the invention can be found isolated as such or forming part of vectors allowing the propagation of said polynucleotides in suitable host cells. Vectors suitable for the insertion of said polynucleotides are vectors derived from expression vectors in prokaryotes such as pUC18, pUC19, Bluescript and the derivatives thereof, mp18, mp19, pBR322, pMB9, Co1E1, pCR1, RP4, phages and “shuttle” vectors such as pSA3 and pAT28, expression vectors in yeasts such as vectors of the type of 2 micron plasmids, integration plasmids, YEP vectors, centromere plasmids and the like, expression vectors in insect cells such as vectors of the pAC series and of the pVL, expression vectors in plants such as pIBI, pEarleyGate, pAVA, pCAMBIA, pGSA, pGWB, pMDC, pMY, pORE series and the like, and expression vectors in eukaryotic cells, including baculovirus suitable for transfecting insect cells using any commercially available baculovirus system. The vectors for eukaryotic cells include preferably viral vectors (adenoviruses, viruses associated to adenoviruses such as retroviruses and, particularly, lentiviruses) as well as non-viral vectors such as pSilencer 4.1-CMV (Ambion), pcDNA3, pcDNA3.1/hyg, pHMCV/Zeo, pCR3.1, pEFI/His, pIND/GS, pRc/HCMV2, pSV40/Zeo2, pTRACER-HCMV, pUB6/V5-His, pVAX1, pZeoSV2, pCI, pSVL and PKSV-10, pBPV-1, pML2d and pTDT1.

The vectors may also comprise a reporter or marker gene which allows identifying those cells that have been incorporated the vector after having been put in contact with it. Useful reporter genes in the context of the present invention include lacZ, luciferase, thymidine kinase, GFP and on the like. Useful marker genes in the context of this invention include, for example, the neomycin resistance gene, conferring resistance to the aminoglycoside G418; the hygromycinphosphotransferase gene, conferring resistance to hygromycin; the ODC gene, conferring resistance to the inhibitor of the ornithine decarboxylase (2-(difluoromethyl)-DL-ornithine (DFMO); the dihydrofolatereductase gene, conferring resistance to methotrexate; the puromycin-N-acetyl transferase gene, conferring resistance to puromycin; the ble gene, conferring resistance to zeocin; the adenosine deaminase gene, conferring resistance to 9-beta-D-xylofuranose adenine; the cytosine deaminase gene, allowing the cells to grow in the presence of N-(phosphonacetyl)-L-aspartate; thymidine kinase, allowing the cells to grow in the presence of aminopterin; the xanthine-guanine phosphoribosyltransferase gene, allowing the cells to grow in the presence of xanthine and the absence of guanine; the trpB gene of E. coli, allowing the cells to grow in the presence of indol instead of tryptophan; the hisD gene of E. coli, allowing the cells to use histidinol instead of histidine. The selection gene is incorporated into a plasmid that can additionally include a promoter suitable for the expression of said gene in eukaryotic cells (for example, the CMV or SV40 promoters), an optimized translation initiation site (for example, a site following the so-called Kozak's rules or an IRES), a polyadenylation site such as, for example, the SV40 polyadenylation or phosphoglycerate kinase site, introns such as, for example, the beta-globulin gene intron. Alternatively, it is possible to use a combination of both the reporter gene and the marker gene simultaneously in the same vector.

The interfering RNAs for use in the present invention are targeted to CD40. The term “CD40” as used herein refers to a 45- to 50-kDa type I integral membrane glycoprotein also known as tumour necrosis factor receptor superfamily member 5 (TNFRSF5). This receptor has been found to be essential in mediating a broad variety of immune and inflammatory responses including T cell-dependent immunoglobulin class switching, memory B cell development, and germinal center formation.

Human CD40 gene is deposited in GenBank (version dated Mar. 12^{th} 2011) with accession number NG_{—}007279.1. Two transcripts are deposited in GenBank for the human CD40. mRNA transcript 1 (mRNA1) is the transcript variant of human CD40 that encodes the longer isoform of 1,616 bp or isoform 1. This mRNA1 is deposited in GenBank with accession number NM_{—}001250.4 mRNA transcript 2 (mRNA2), is a transcript variant of human CD40 of 1554 pb that lacks a coding segment, which leads to a translation frame shift, compared to variant mRNA1. The resulting iso form 2 contains a shorter and distinct C-terminus, compared to iso form 1. The mRNA2 is deposited in GenBank with accession number NM_{—}152854.2. Two human protein isoforms are deposited in GenBank: isoform 1 (NP_{—}001241.1) of 277 amino acids and isoform 2 (NP_{—}690593.1) of 203 amino acids.

The interfering RNAs according to the present invention may be targeted to any region of the CD40 mRNA provided that an effective silencing is achieved. Methods for determining the degree of silencing of the CD40 mRNA have been described above. In a preferred embodiment, the interfering RNAs are targeted to the regions in the CD40 mRNA corresponding to positions 173-193, 192-212, 479-499, 709-729, 62-82, 137-157, 214-234, 242-262 or 188-214 of the human CD40 mRNA wherein the numbering corresponds to the position with respect to the start codon in the CD40 cDNA as defined in NCBI accession X60592.1.

In a preferred embodiment, the siRNAs are those shown in Table 1.

TABLE 1

| Targeted | | |
|---|---|---|
| region | | SEQ |
| in human | | ID |
| CD40 mRNA | Sequence | NO |
| 173-193 | 5′-UGCCUUCCUUGCGGUGAAA-3′ | 1 |
| | 5′-UUUCACCGCAAGGAAGGCA-3′ | 2 |
| 192-212 | 5′-GCGAAUUCCUAGACACCUG-3′ | 3 |
| | 5′-CAGGUGUCUAGGAAUUCGC-3′ | 4 |
| 479-499 | 5′-UGUCACCCUUGGACAAGCU-3′ | 5 |
| | 5′-UGCUUGUCCAAGGGUGACA-3′ | 6 |
| 709-729 | 5′-UUUUCCCGACGAUCUUCCU-3′ | 7 |
| | 5′-AGGAAGAUCGUCGGGAAAA-3′ | 8 |
| 62-82 | 5′-CCACCCACUGCAUGCAGAG-3′ | 9 |
| | 5′-CUCUGCAUGCAGUGGGUGG-3′ | 10 |
| 137-157 | 5′-CUGGUGAGUGACUGCACAG-3′ | 11 |
| | 5′-CUGUGCAGUCACUCACCAG-3′ | 12 |
| 214-234 | 5′-CAGAGAGACACACUGCCAC-3′ | 13 |
| | 5′-GUGGCAGUGUGUCUCUCUG-3′ | 14 |
| 242-262 | 5′-UACUGCGACCCCAACCUAG-3′ | 15 |
| | 5′-CUAGGUUGGGGUCGCCAGUA-3′ | 16 |
| 188-214 | 5′-GAAAGCGAAUUCCUAGACACCUGGAAC-3′ | 17 |
| | 5′-GUUCCAGGUGUCUAGGAAUUCGCUUUC-3′ | 18 |

Sequence composition and target localization within human CD40 mRNA of siRNAs designed to screen for efficient CD40 mRNA silencing. Numbering is provided from the ATG start codon in the CD40 mRNA as shown in NCBI accession number X60592 (version 1 of 14 NOV. 1997)

Other illustrative, non-limitative, examples of interfering RNA specific for the sequence of CD40 include the mouse CD40 siRNA sc-29998, the mouse CD40 shRNA plasmid sc-29998-SH, the mouse CD40 shRNA lentiviral particles sc-29998-V, the human CD40 siRNA sc-29250, the human shRNA plasmid sc-29250-SH and the human CD40 shRNA lentiviral particles sc-29250-V, all of them from Santa Cruz Biotechnology and the human CD40 hairpin siRNA eukaryotic expression vectors as in Chen L. & Zheng X X, Chinese J Cell Mol Immunol 2005; 21(2):163-6.

Preferred interfering RNAs targeted to human CD40 gene are those targeted towards a stable internal loop within the secondary structure of the CD40 mRNA.

In a particular embodiment of the invention, the interfering RNA that silences CD40 gene expression is a short interfering RNA (siRNA).

In certain instances, the interfering RNA may be modified by a non-ligand group in order to enhance the activity, cellular distribution or cellular uptake of the dsRNA. Procedures for performing such conjugations are available in the scientific literature. Such non-ligand moieties include lipid moieties, such as cholesterol (Letsinger et al., Proc. Natl. Acad. Sci. USA, 1989, 86:6553), cholic acid (Manoharan et al, Bioorg. Med. Chem. Lett., 1994, 4:1053), a thioether, e.g., hexyl-S-tritylthiol (Manoharan et al, Ann. N.Y. Acad. Sci., 1992, 660:306; Manoharan et al, Bioorg. Med. Chem. Let., 1993, 3:2765), a thiocholesterol (Oberhauser et al, Nucl. Acids Res., 1992, 20:533), an aliphatic chain, e.g., dodecandiol or undecyl residues (Saison-Behmoaras et al., EMBO J., 1991, 10:111; Kabanov et al, FEBS Lett., 1990, 259:327; Svinarchuk et al, Biochimie, 1993, 75:49), a phospholipid, e.g., di-hexadecyl-rac-glycerol or triethylammonium 1,2-di-O-hexadecyl-rac-glycero-3-H-phosphonate (Manoharan et al, Tetrahedron Lett., 1995, 36:3651; Shea et al, Nucl. Acids Res., 1990, 18:3777), a polyamine or a polyethylene glycol chain (Manoharan et al, Nucleosides and Nucleotides, 1995, 14:969), or adamantane acetic acid (Manoharan et al, Tetrahedron Lett., 1995, 36:3651), a palmityl moiety (Mishra et al, Biochim. Biophys. Acta, 1995, 1264:229), or an octadecylamine or hexylamino-carbonyl-oxycholesterol moiety (Crooke et ah, J. Pharmacol. Exp. Ther., 1996, 277:923). Typical conjugation protocols involve the synthesis of the interfering RNA bearing an aminolinker at one or more positions of the sequence. The amino group is then reacted with the molecule being conjugated using appropriate coupling or activating reagents. The conjugation reaction may be performed either with the RNA still bound to the solid support or following cleavage of the RNA in solution phase. Purification of the RNA conjugate by HPLC typically affords the pure conjugate. In some embodiments, an interfering RNA described herein is covalently bound to a lipophilic ligand. Exemplary lipophilic ligands include cholesterol; bile acids; and fatty acids {e.g., lithocholic-oleyl acid, lauroyl acid, docosnyl acid, stearoyl acid, palmitoyl acid, myristoyl acid, oleoyl acid, or linoleoyl acid)

The interfering RNA or the polynucleotide coding for said interfering RNA of the invention can be administered forming part of liposomes, conjugated to cholesterol or conjugated to compounds capable of causing the translocation through cell membranes such as the TAT peptide, derived from the HIV-1 TAT protein, the third helix of the homeodomain of the D. melanogaster Antennapaedia protein, the VP22 protein of the herpes simplex virus, arginine oligomers and peptides such as those described in WO07069090 (Lindgren, A. et al., 2000, Trends Pharmacol. Sci., 21:99-103; Schwarze, S. R. et al., 2000, Trends Pharmacol. Sci., 21:45-48, Lundberg, M. et al., 2003, Mol. Therapy, 8:143-150 and Snyder, E. L. and Dowdy, S. F., 2004, Pharm. Res., 21:389-393).

In a particular embodiment, the interfering RNA or the polynucleotide coding for said interfering RNA of the invention are administered by means of the so-called “hydrodynamic administration” in which the interfering RNA or the polynucleotide coding for said interfering RNA are introduced intravascularly into the organism at high speed and volume, which results in high transfection levels with a more diffuse distribution (Aliño, S. F. et al. 2010. J Gene Med, 12:920-6). A modified version of this technique has made it possible to obtain positive results for silencing through the naked siRNAs of exogenous genes (Lewis et al., 2002, Nat. Gen., 32:107-108; McCaffrey et al., 2002, Nature, 418:38-39) and endogenous genes (Song et al., 2003, Science, Nat. Med., 9:347-351) in multiple organs. It has been shown that the effectiveness of the intercellular access depends directly on the volume of the fluid administered and the speed of the injection (Liu et al., 1999, Science, 305:1437-1441). In mice, the administration has been optimized at values of 1 ml/10 g of body weight in a period of 3-5 seconds (Hodges, et al., 2003, Exp. Opin. Biol. Ther., 3:91-918). The exact mechanism allowing in vivo cell transfection with siRNAs after their hydrodynamic administration is not fully known. In the case of mice, it is thought that administration through the tail vein takes place at a rate that exceeds the heart rate and that the administrated fluid accumulates in the superior vena cava. This fluid subsequently accesses the vessels in the organs, and after that, through fenestrations in said vessels, accesses the extravascular space. In this way, the siRNA comes into contact with the cells of the target organism before it is mixed with the blood, thus reducing the possibilities of degradation through nucleases.

Alternatively, the interfering RNA or the polynucleotide coding for said interfering RNA of the invention may be administered forming part of polyplexes which are complexes of polymers with DNA. Most polyplexes consist of cationic polymers and their production is regulated by ionic interactions. One large difference between the methods of action of polyplexes and lipoplexes is that polyplexes cannot release their DNA load into the cytoplasm, so to this end, co-transfection with endosome-lytic agents (to lyse the endosome that is made during endocytosis, the process by which the polyplex enters the cell) such as inactivated adenovirus must occur. However, this is not always the case, polymers such as polyethylenimine have their own method of endosome disruption as does chitosan and trimethylchitosan.

Alternatively, the interfering RNA or the polynucleotide coding for said interfering RNA of the invention can be administered associated to dendrimers which are repeatedly branched, roughly spherical large molecules capable of delivering the oligonucleotides.

In a particular embodiment, the interfering RNA of the invention is administered subcutaneously, intradermally, intramuscularly, intraocularly, intrathecally, intracerebellarly, intranasally, intratracheally, hypodermically, intraperitoneally, intrahepatically, intratesticularly, intratumorally, hypodermically, by injection or by intravascular perfusion.

The amount of interfering RNA or the polynucleotide coding for said interfering RNA required for the therapeutic or prophylactic effect will naturally vary according to the elected interfering RNA or polynucleotide coding for said interfering RNA, the nature and the severity of the illness to be treated, and the patient. A specific dosage and treatment regimen for any particular patient will depend upon a variety of factors, including the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

Use of Interfering siRNA Specific for CD40 for the Treatment of Ischemia/Reperfusion Injury

The authors of the present invention have found that the silencing of CD40 gene expression by RNA interference results in the significant functional benefits according to urea and creatinine levels (see example 2).

Thus, in a second aspect, the invention relates to an interfering RNA which silences CD40 gene expression or a polynucleotide coding for said interfering RNA for use in the prevention or treatment of ischemia/reperfusion injury.

Alternatively, the invention relates to the use of an interfering RNA that silences CD40 gene expression or a polynucleotide coding for said interfering RNA for the manufacture of a medicament for the treatment of ischemia/reperfusion injury.

Alternatively, the invention relates to a method for the prevention and/or the treatment of ischemia/reperfusion injury in a subject in need thereof which comprises the administration to said subject of an interfering RNA that silences CD40 gene expression or a polynucleotide coding for said interfering RNA.

The term “ischemia/reperfusion injury”, as used herein, refers to tissue damage caused when blood supply returns to the tissue after a period of ischemia. The absence of oxygen and nutrients from blood during the ischemic period creates a condition in which the restoration of circulation results in inflammation and oxidative damage through the induction of oxidative stress rather than restoration of normal function. Oxidative stresses associated with reperfusion may cause damage to the affected tissues or organs. Ischemia-reperfusion injury is characterized biochemically by a depletion of oxygen during an ischemic event followed by reoxygenation and the concomitant generation of reactive oxygen species during reperfusion (Piper, H. M., Abdallah, C, Schafer, C, Annals of Thoracic Surgery 2003, 75:644; Yellon, D. M., Hausenloy, D. J., New England Journal of Medicine 2007, 357:1121).

An ischemia-reperfusion injury can be caused, for example, by a natural event (e.g., restoration of blood flow following a myocardial infarction), a trauma, or by one or more surgical procedures or other therapeutic interventions that restore blood flow to a tissue or organ that has been subjected to a diminished supply of blood. Such surgical procedures include, for example, coronary artery bypass graft surgery, coronary angioplasty, organ transplant surgery and the like (e.g., cardiopulmonary bypass surgery). For the treatment of ischemic and ischemia-reperfusion injuries caused by therapeutic interventions, such as surgical procedures, it is preferable that a compound of the invention is administered to a subject undergoing treatment prior to the therapeutic intervention (e.g., cardiac surgery, organ transplant). For example, a compound of the invention can be administered to a subject undergoing treatment, e.g., about 1 hour, about 2 hours, about 3 hours, about 4 hours, about 5 hours, about 12 hours, about 24 hours, or about 48 hours prior to the therapeutic intervention. A compound of the invention can also be administered to a subject undergoing treatment, for example, about 5 minutes, about 10 minutes, about 15 minutes, about 20 minutes, about 30 minutes or about 45 minutes prior to the therapeutic intervention.

A compound of the invention can also be used to inhibit an ischemia or ischemia-reperfusion injury to a cell, tissue or organ, ex vivo, prior to a therapeutic intervention (e.g., a tissue employed in a graft procedure, an organ employed in an organ transplant surgery). For example, prior to transplant of an organ into a host individual (e.g., during storage or transport of the organ in a sterile environment), the organ can be contacted with a compound of the invention (e.g., bathed in a solution comprising a compound of the invention) to inhibit ischemia or ischemia-reperfusion injury.

The terms “treatment”, “prevention”, “subject”, “interfering RNA that silences CD40 gene expression” and “polynucleotide coding for said interfering RNA” have been described in detail above and are used with the same meaning in the context of the present method.

In a preferred embodiment, the interfering RNA is targeted to a region in the CD40 mRNA selected from the group consisting of a region located at positions 173-193, 192-212, 479-499, 709-729, 62-82, 137-157, 214-234, 242-262 or 188-214 of the human CD40 mRNA wherein the numbering corresponds to the position with respect to the start codon in the CD40 cDNA as defined in NCBI accession X60592.1. In a still more preferred embodiment, the interfering RNA for use as in any of claims 1 to 2 wherein said interfering RNA comprises a sequence as defined in Table 1.

In another embodiment, the interfering RNA is a short interfering RNA (siRNA). The interfering RNA may be administered subcutaneously, intradermally, intramuscularly, intraocularly, intrathecally, intracerebellarly, intranasally, intratracheally, hypodermically, intraperitoneally, intrahepatically, intratesticularly, intratumorally, hypodermically, by injection or by intravascular perfusion.

Use of Interfering siRNA Specific for CD40 for the Treatment of Sepsis

The authors of the present invention have also observed that the administration of interferring siRNAs specific for CD40 is capable of attenuating the increased in expression of CD40 resulting from LPS exposure (see example 3). Since LPS is one of the factors causative of sepsis, the interference of the expression of CD40 by the use of interfering RNA is also useful for the treatment or prevention of sepsis or for the treatment or prevention of the symptoms of sepsis caused by LPS.

Thus, in a third aspect, the invention relates to an interfering RNA which silences CD40 gene expression or a polynucleotide coding for said interfering RNA for use in the prevention or treatment of sepsis.

Alternatively, the invention relates to the use of an interfering RNA that silences CD40 gene expression or a polynucleotide coding for said interfering RNA for the manufacture of a medicament for the treatment of sepsis.

Alternatively, the invention relates to a method for the prevention and/or the treatment of sepsis injury in a subject in need thereof which comprises the administration to said subject of an interfering RNA that silences CD40 gene expression or a polynucleotide coding for said interfering RNA.

The term “sepsis”, as used herein, refers to a condition defined as “a Systemic Inflammatory Response Syndrome (SIRS) secondary to infection”. Such a condition is characterized by a manifested infection induced by microorganisms, preferably bacteria or fungi, by parasites or by viruses or prions. The term comprises different forms of sepsis, e.g. urosepsis, sepsis due to pneumonia, intraabdominal infection, postoperative sepsis, sepsis due to invasion of a foreign body, sepsis due to bone marrow insufficiency or neutropenia, cholangiosepsis, sepsis after skin injury, burn or dermatitis, dentogenic or tonsillogenic sepsis. As used herein, the terms “sepsis” and “septic syndrome” are equivalent and interchangeable.

The more general term SIRS describes a generalized hyper-inflammatory reaction of diverse geneses, e.g. infection, burn and trauma. Thus, “sepsis” is a particular form of SIRS, namely a SIRS characterized by infection of normally or physiologically sterile tissue.

Particular forms of the condition of sepsis are “severe sepsis” and “septic shock”. “Severe sepsis” is defined as “a sepsis associated with (multiple) organ dysfunction, hypoperfusion, or hypotension”. “Septic shock” is defined as a sepsis with hypotension, despite fluid resuscitation, along with the presence of perfusion abnormalities.

In one embodiment, the sepsis syndrome is induced by LPS.

In one embodiment, the condition associated with sepsis syndrome is selected from the group consisting of an organ dysfunction, preferably a kidney dysfunction or a liver dysfunction, a multiple organ dysfunction syndrome (MODS), an acute respiratory distress syndrome (ARDS), and disseminated intravascular coagulation (DIC). In another embodiment, the sepsis syndrome is induced by a bacterium or more than one bacterium selected from the group consisting of Gram-negative bacteria and Gram-positive bacteria. In yet another embodiment, the Gram-negative bacterium is selected from the group consisting of Escherichia coli, Klebsiella species, Serratia species, Enterobacter species, Proteus species, Pseudomonas aeruginosa, Haemophilus influenzae, Neisseria species, and Listeria species. In another embodiment, the Gram-positive bacterium is selected from the group consisting of Staphylococcus aureus, Streptococcus pneumoniae, coagulase-negative Staphylococci, Enterococcus species, Streptococcus pyogenes, and Streptococcus viridans. In one embodiment, the bacterium is a Gram-negative bacterium, preferably E. coli. In one embodiment, the sepsis syndrome is induced by a microorganism or more than one microorganism selected from the group consisting of anaerobic bacteria, fungi, rickettsiae, chlamydiae, mycoplasma, spirochetes, and viruses.

The terms “treatment”, “prevention”, “subject”, “interfering RNA that silences CD40 gene expression” and “polynucleotide coding for said interfering RNA” have been described in detail above and are used with the same meaning in the context of the present method.

In a preferred embodiment, the interfering RNA is targeted to a region in the CD40 mRNA selected from the group consisting of a region located at positions 173-193, 192-212, 479-499, 709-729, 62-82, 137-157, 214-234, 242-262 or 188-214 of the human CD40 mRNA wherein the numbering corresponds to the position with respect to the start codon in the CD40 cDNA as defined in NCBI accession X60592.1. In a still more preferred embodiment, the interfering RNA for use as in any of claims 1 to 2 wherein said interfering RNA comprises a sequence as defined in Table 1.

In another embodiment, the interfering RNA is a short interfering RNA (siRNA). The interfering RNA may be administered subcutaneously, intradermally, intramuscularly, intraocularly, intrathecally, intracerebellarly, intranasally, intratracheally, hypodermically, intraperitoneally, intrahepatically, intratesticularly, intratumorally, hypodermically, by injection or by intravascular perfusion.

In one embodiment, the subject is a mammal, preferably a human.

The invention is described in detail below by means of the following examples which are to be construed as merely illustrative and not limitative of the scope of the invention.

### EXAMPLES

### Example 1

### Use of CD40 siRNA in the Treatment of Murine Lupus Nephritis

### Animals

Animals NZB/W F1 female mice (Charles River, Spain) were used. The experiment was carried out in accordance with current legislation on animal experiments in the European Union and approved by our institution's Ethics Committee for Animal Research. Mice were housed in a constant temperature room with a 12-hour dark/12-hour light cycle, and were given free access to water and a standard laboratory diet.

### Study Design and Follow-Up

Five-month old NZB/W F1 female mice were divided into the following groups:
CYP (n=8)—mice were treated with intraperitoneal CYP 50 μg/kg every 10 days;
CTLA4-Ig (n=9)—mice were treated with intraperitoneal 50 μg CTLA4-Ig three times a week.
siRNA 1/w (n=9): mice were treated intraperitoneally with 50 μg of murine specific anti CD40 siRNA once a week;
siRNA 2/w P (n=9): mice were treated intraperitoneally with 50 μg of murine specific anti CD40 siRNA twice a week;
Control (n=11)—non-treated animals.

Treatment was given from six to nine old months. Body weight was determined twice a month. Mice were placed in metabolic cages to collect 24-hour urine specimens before treatment, and again at six, seven and eight months. Blood was obtained from the tail vein at the same intervals. At the end of the follow-up, survivor animals were sacrificed and kidney processed for histological studies.

### Proteinuria and Renal Function

Urinary protein concentration was determined by a commercial kit based on the Ponceau method (BayerDiagnostics, Madrid, Spain). Serum creatinine levels (in milligrams per decilitre) were determined by Jaffés method on an autoanalyzer (Beckman Instruments, Palo Alto, Calif.) at the end of the follow-up. Anti-DNA antibodies levels of anti-DNA antibodies were measured before treatment, and subsequently each month and at sacrifice, using a commercially available ELISA kit (Alpha Diagnostic International, San Antonio, Tex., USA) according to manufacturer's instructions (serum diluted 1:100). This kit is based on a purified dsDNA coated microwell plate. Antibodies to dsDNA are directed against the phosphate-deoxyribose backbone of the DNA molecule. Anti-dsDNA from serum samples bind to extracted nuclear antigen immobilized on microtiter wells. Anti-mouse IgG-HRP conjugate is added, and the colour developed by chromogenic substrate addition. The enzymatic reaction (blue colour) is directly proportional to the amount of the anti-dsDNA in the sample. Kits contain positive and negative controls, and anti-DNA antibodies are estimated in a semi-quantitative way according to the optical density (o.d. 450).

### Histological Studies

For histological studies, 1-2-mm-thick coronal slices of kidney were fixed in 4% formaldehyde and embedded in paraffin. For light microscopy 3-4 mm thick tissue sections were stained with hematoxylin and eosin (H&E), periodic acid-Schiff and Masson's trichrome. All renal biopsies were analyzed by two blinded pathologists. Typical active lesions of lupus nephritis (mesangial expansion, endocapillary proliferation, glomerular deposits, extracapillary proliferation and interstitial infiltrates), as well as chronic lesions (tubular atrophy and interstitial fibrosis) were evaluated. Lesions were graded semi-quantitatively using a scoring system from 0 to 3 (0=no changes, 1=mild, 2=moderate, 3=severe). Finally, a total histological score (HS) was derived from the sum of all the described items. Renal immunofluorescence. For analysis of Ig and complement deposition, fluorescent staining of cryosections was used. Five-micrometer cryostat sections were fixed in acetone at −80° C. for 20 minutes, then blocked with 20% normal goat serum and directly stained with FITC-conjugated goat anti-mouse IgG at 1/300 (Sigma-Aldrich). To stain for complement, slides were then sequentially incubated in rat anti-mouse complement C3, at 1/50 (Cedarlane Laboratories Limited), followed by Alexa Fluor 546-conjugated goat anti-rat IgG at 1/1000 (Invitrogen, Molecular Probes). At least 30 glomeruli, visualized and photographed with an immunofluorescence microscope, from each animal were examined in a binded manner. A semiquantitative score of staining intensity and distribution from 0 to 4+ was given, where 0+ denotes no signal; 1+, minimal signal; 2+, low signal; 3+, moderate signal and 4+, strong signal intensity.

### Statistical Analysis

Data are expressed as mean+SE. Overall survival was analyzed by the Kaplan-Meier method. One-way analysis of variance (ANOVA) with post hoc tests was performed to compare proteinuria, anti-DNA antibodies and serum creatinine throughout the follow-up. To compare histological data, the non-parametric Kruskal-Wallis test was used. All P-values were two-tailed, and a P-value <0.05 was considered significant.

### Results

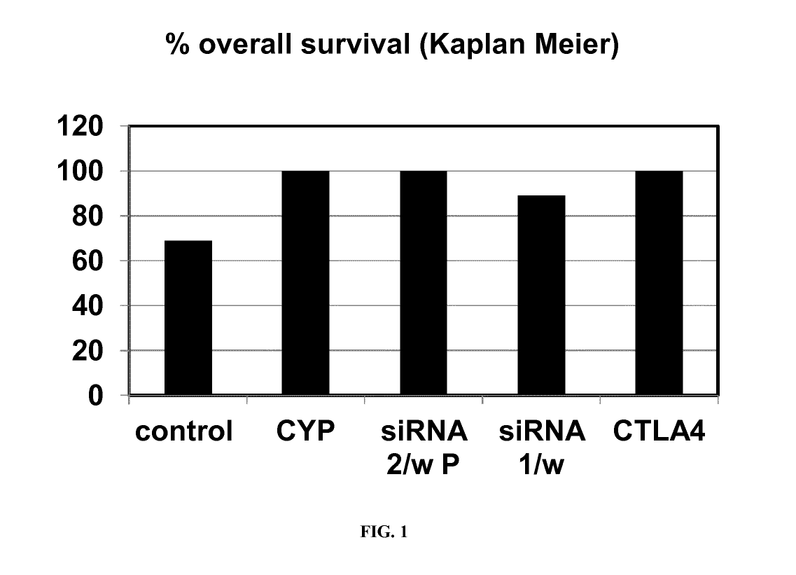

Animal survival (%) was not significantly different among the 3 groups with drug therapy, as shown in FIG. 1. Cumulative survival analyzed by the Kaplan-Meier method was 72% for the control group, 100% for CYP, CTLA-4 Ig and siRNA-CD40 2 doses/week groups, and 89% for the siRNA-CD40 1 dose/week group.

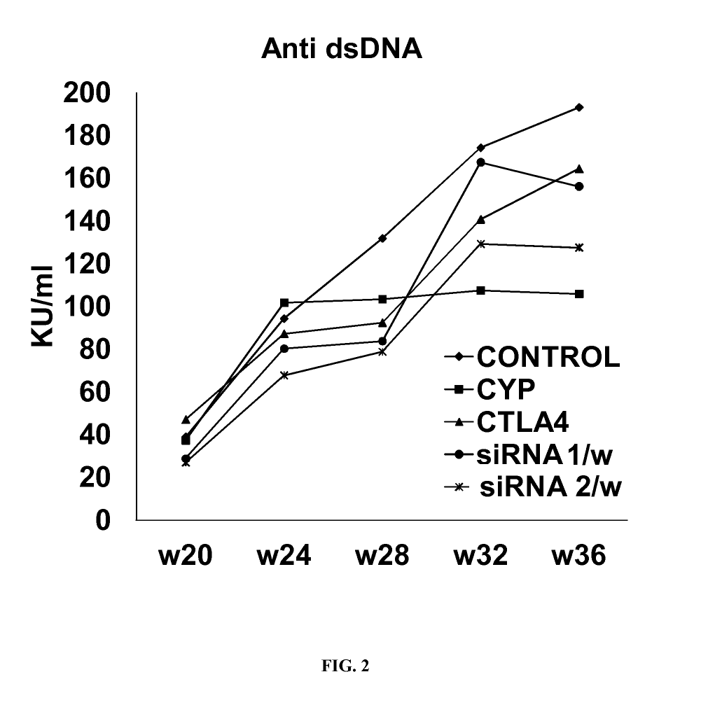

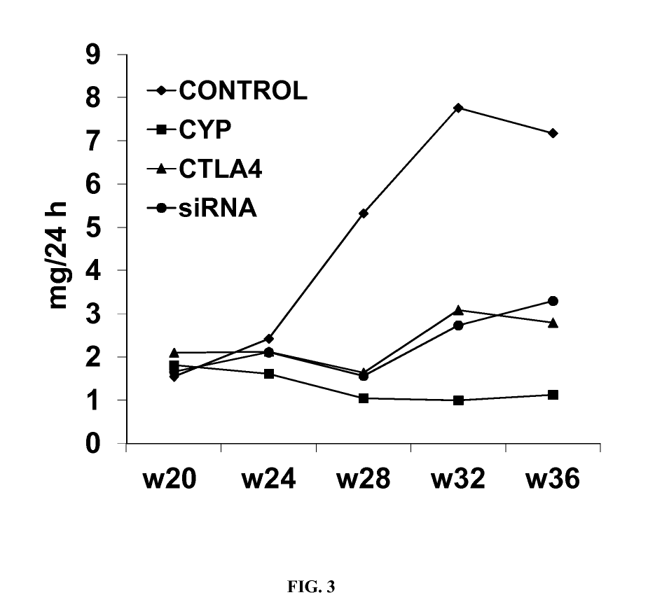

As shown in FIG. 2, anti-dsDNA titers were lower in the CYP group. FIG. 3 shows that proteinuria was significantly lower in CYP group vs. control and intermediate in CTLA4-Ig and siRNA-CD40 groups.

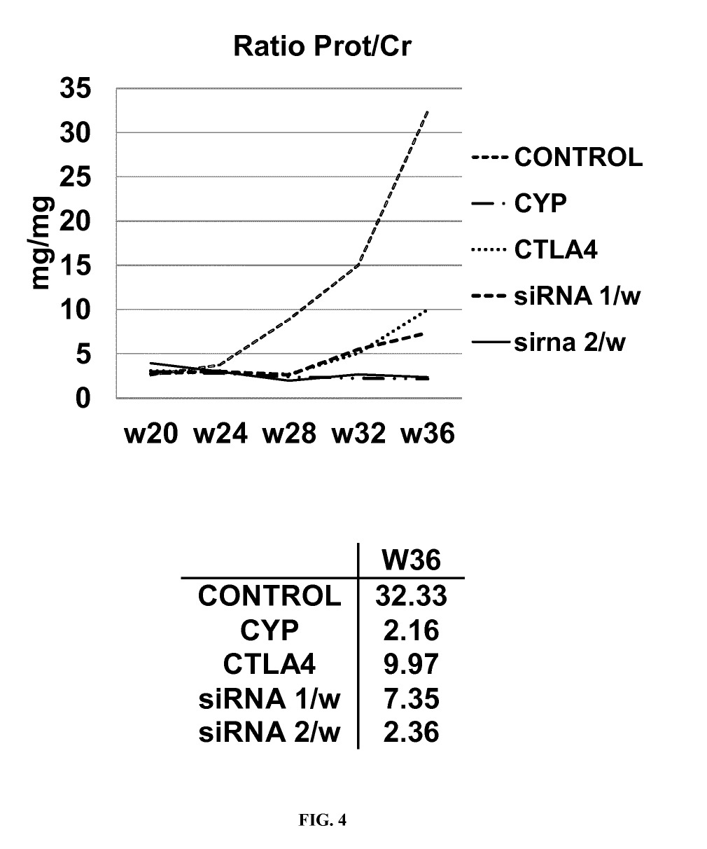

FIG. 4 shows the decrease in the ratio of proteinuria and creatinine levels in the groups analyzed. The ratio for the siRNA 2 doses/week group was similar to that observed for the CYP group.

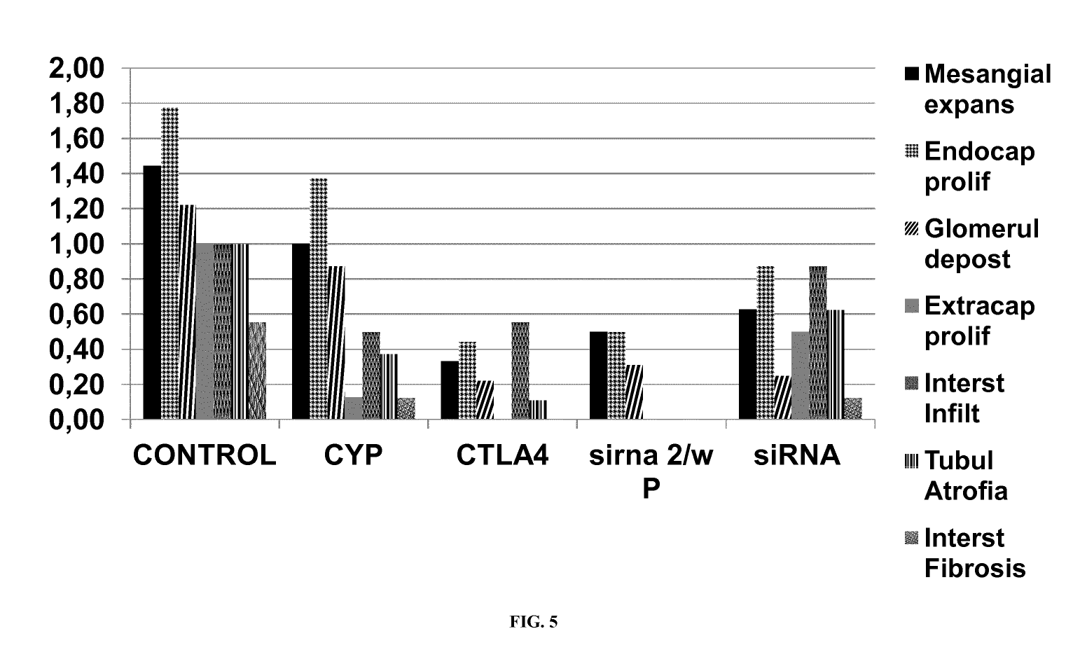

The main histological findings assessed in a semi-quantitative scale showed that costimulatory blockade reduced the main elementary histological lesions of lupus nephritis. As shown in FIG. 5, CD40 gene silencing attenuated acute lesions such as endocapillary proliferation, mesangial expansion, extracapillary proliferation and glomerular deposits, and chronic lesions of tubular atrophy and interstitial fibrosis.

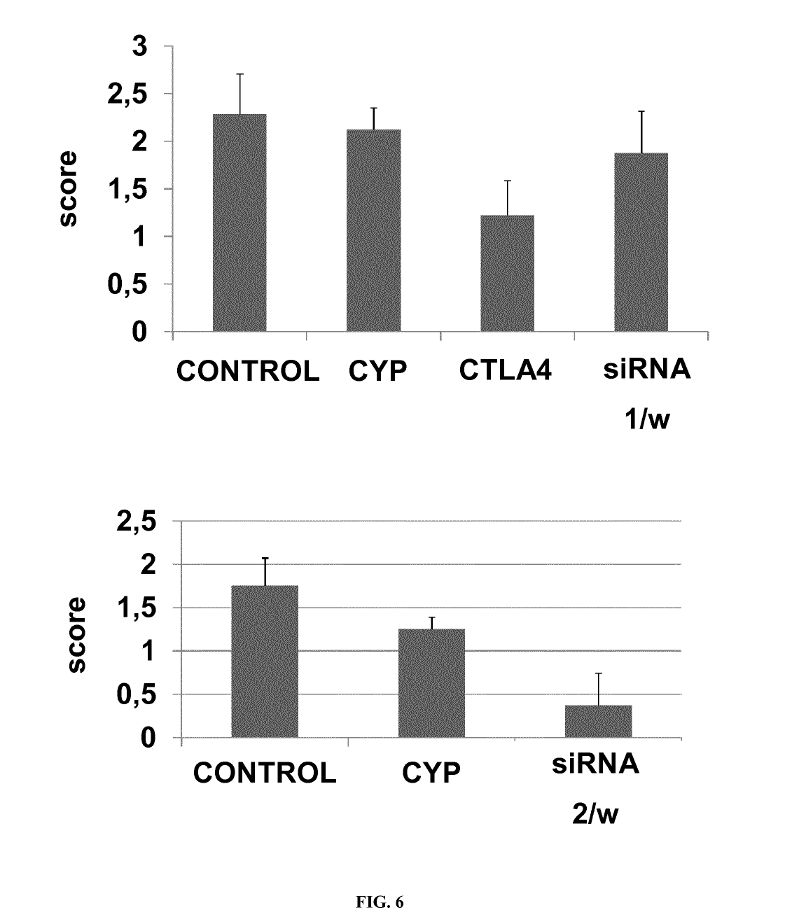

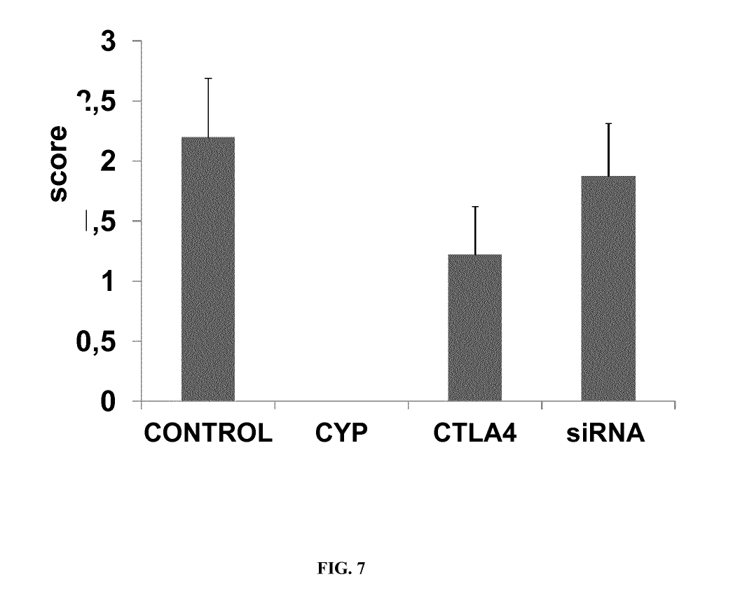

The FIGS. 6 and 7 show glomerular deposition of IgG and C3, respectively. The effect of CTLA4-Ig and siRNA-CD40 on IgG deposition was similar to CYP, although this agent was more effective in reducing C3 deposition than costimulatory blockade.

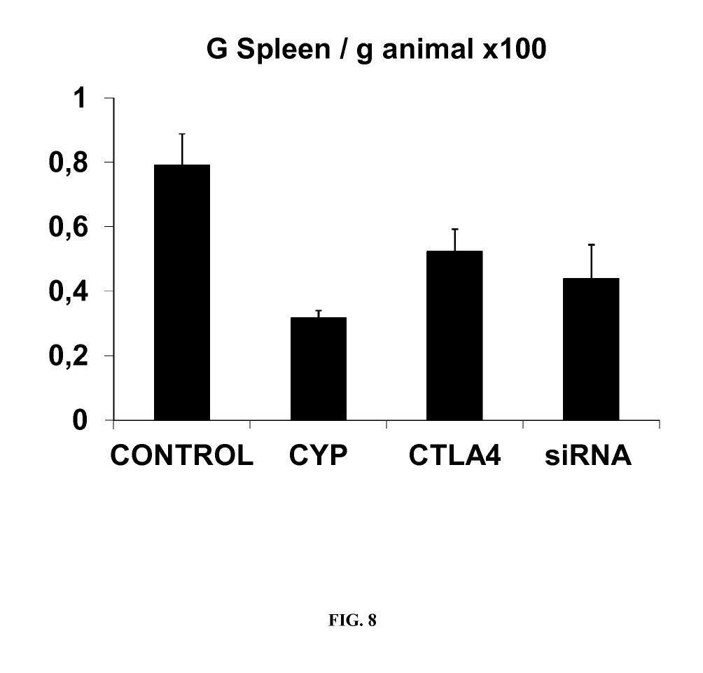

As shown in FIG. 8, all treated animals had smaller spleens than control animals.

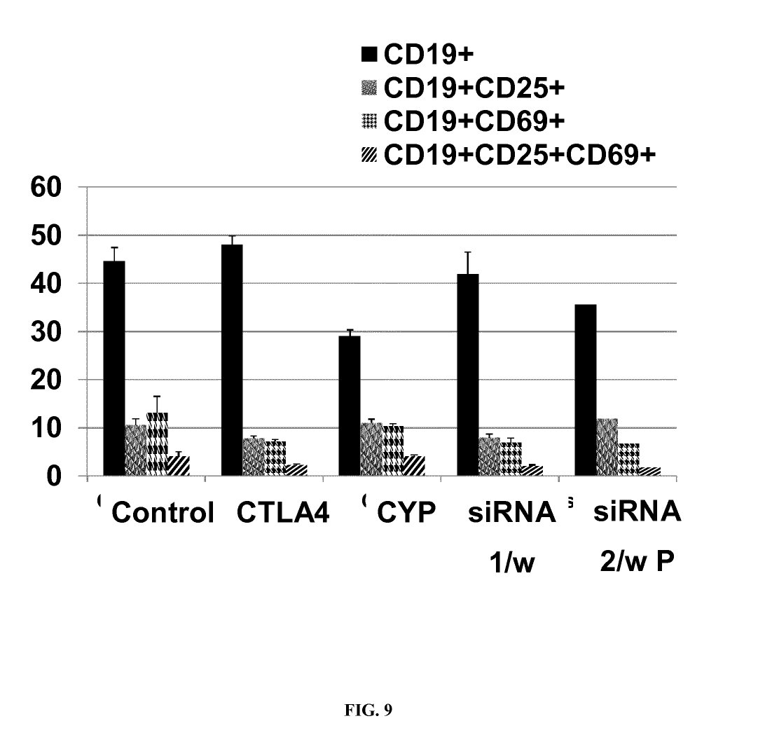

The proportion of B cells in splenocytes was markedly reduced in CYP-treated animals (FIG. 9). In contrast, costimulatory blockade with CTLA4-Ig or siRNA-CD40 reduced the proportion of activated B cells.

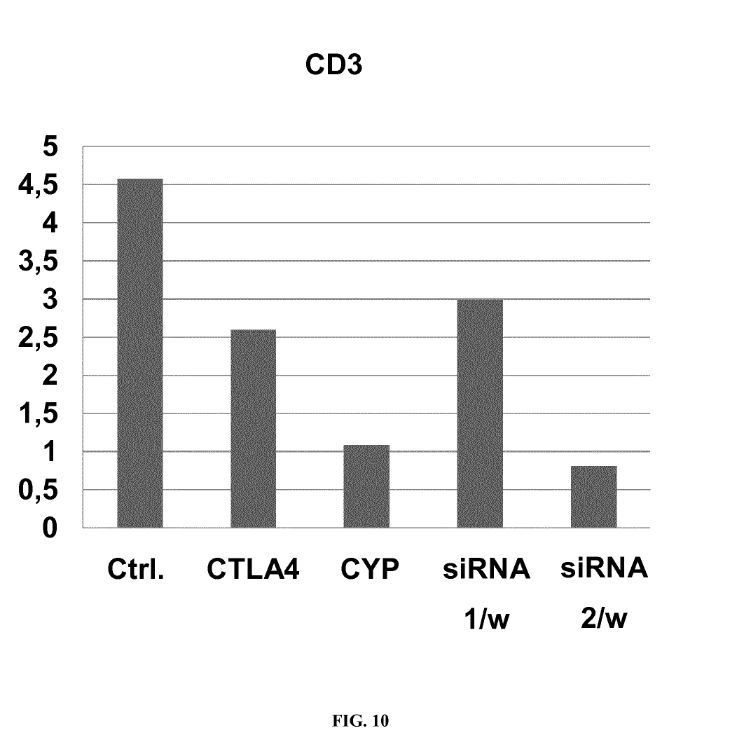

The number of intra-renal CD3+ T cells is shown in FIG. 10.

Thus, from these data the inventors concluded that:
siRNA-CD40 attenuated histological lesions of murine lupus nephritis to similar levels of other costimulatory blockers.
CD40 gene silencing has no clear effect on anti-dsDNA antibodies, although reduces B cell activation and
Intensified siRNA-CD40 dosage reduces proteinuria to similar levels to the gold-standard therapy using cyclophosphamide.

### Example 2

### Use of siRNA-CD40 in the Prevention of Renal Ischemia/Reperfusion Injury

The CD40-siRNA development program was aimed at reducing the risk of delayed graft function (DGF) in patients undergoing deceased donor renal transplantation, where the unavoidable cold storage ischemia is followed by reperfusion injury, which induces renal parenchymal damage and functional impairment.

In order to assess the localization of naked siRNA-CD40-Chol in the renal parenchyma, this agent (50 μg) labelled with Cy 5.5 was injected intravenously (i.v) and the fluorescence of renal tissue quantified at several time intervals.

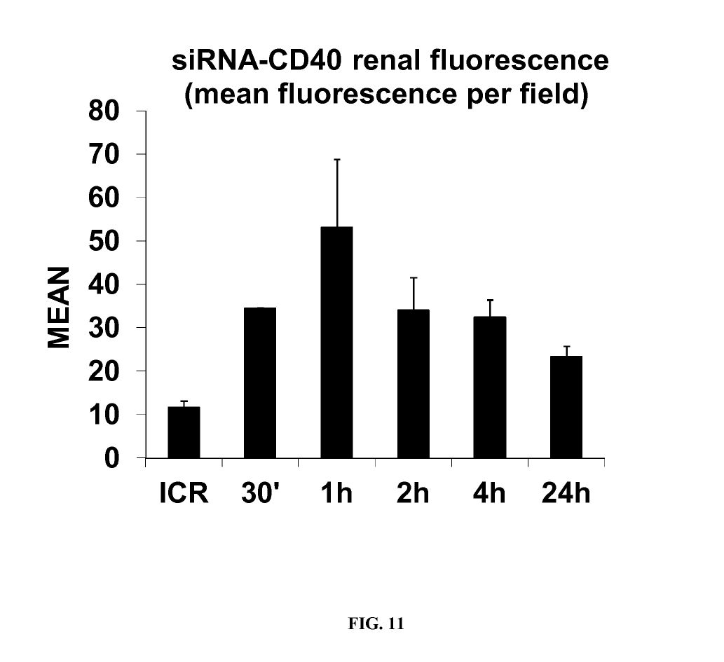

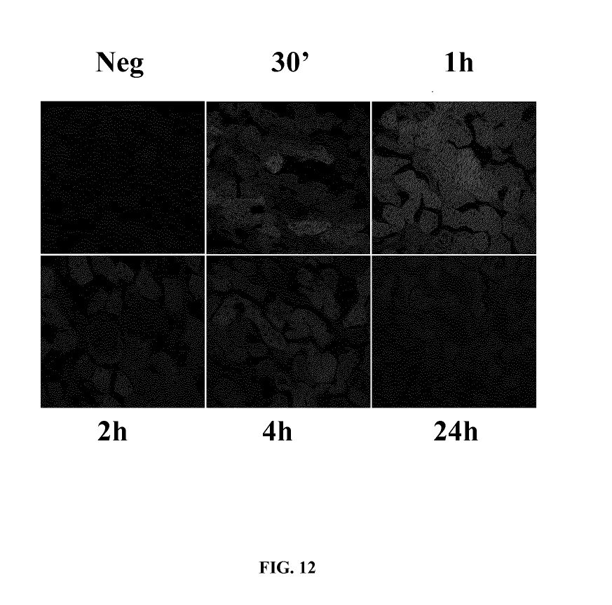

In comparison with ICR non-injected mice, after the i.v. administration of labelled siRNA-CD40, fluorescence peaked at 1 hour a gradually decreased to 24 hours (FIG. 11). siRNA-CD40 was mainly localized in renal tubules (FIG. 12).

Taking into account the intense localization of siRNA-CD40 in renal tissue after i.v. injection new experiments of renal warm ischemia were conducted in rodents (rats and mice).

In anesthetized animals the two kidneys was surgically exposed with a midline laparotomy. The renal arteries and veins were clamped in block to induce kidney ischemia for 45 min, during which animals were kept at 37° C. Then, clamps were released and the animals were housed for a week. Animals were divided into 2 groups: wIRI control group: 45 min of warm ischemia and no treatment; wIRI-siRNA-CD40 group with warm ischemia and intravenous injection of 50 μg of siRNA-CD40 immediately before vascular de-clamping. On days 1, 3, 5 and 7 the animals were weighed and blood obtained from tail vein, used for measuring creatinine and urea levels (mg/dl). Kidneys were processed for histological and molecular studies.

These data show significant functional benefits of siRNA-CD40 treatment in both species according to urea and creatinine levels (Table 2).

TABLE 2
Creatinine and urea levels in the serum of rats and mice

| | | Serum creatinine | | Serum urea | |
|---|---|---|---|---|---|
| | | (mg/dl) | | (mg/(dl) | |
| Rats | N | 24 h | 48 h | 24 h | 48 h |
| wIRI (controls) | 7 | 1.95 ± 0.23 | 1.15 ± 0.13 | 185.5 ± 22.6 | 110.8 ± 18.4 |
| wRI-siRNA- | 7 | 0.71 ± 0.07 | 0.58 ± 0.04 | 48.4 ± 6.2 | 40.9 ± 2.9 |
| CD40 | | | | | |
| p | | 0.0003 | 0.0012 | <0.0001 | 0.0028 |
| Mice | N | | 48 h | | 48 h |
| wIRI (controls) | 5 | | 1.66 ± 0.43 | | 440.4 ± 73.5 |
| wIRI-siRNA- | 6 | | 0.35 ± 0.07 | | 98.4 ± 31.7 |
| CD40 | | | | | |
| p | | | 0.0095 | | 0.0014 |

### Example 3

### The Effect of siRNA-CD40 in the Reduction of CD40 Tissue Expression in a Murine Model of LPS Toxicity

The potency and duration of CD40 gene silencing was addressed in an animal model of LPS toxicity due to the high levels of CD40 expression induced in this model. In order to assess the ability of murine siRNA-CD40 to reduce the expression of CD40 after LPS injection several experimental groups were established. ICR mice received a single initial siRNA-CD40 dose and subsequent LPS injections after different time intervals, as follows:
Groups:
1. Untreated control ICR
2. Day 0+Sac LPS 4 h
3. ip naked siRNA. Day 0+0+LPS Sac 0 h 4 h Day Day 0
4. ip naked siRNA. Day 0+Day 1+LPS 0 h 4 h Sac Day 1
5. ip naked siRNA. Day 0+Day 3+LPS 0 h 4 h Sac Day 3
6. ip naked siRNA. Day 0+5+LPS Sac 0 h 4 h Day Day 5
7. ip naked siRNA. Day 0+Day 7+LPS 0 h 4 h Sac Day 7
8. ip naked siRNA. Day 0+LPS Sac 0 h 4 h Day 14+Day 14

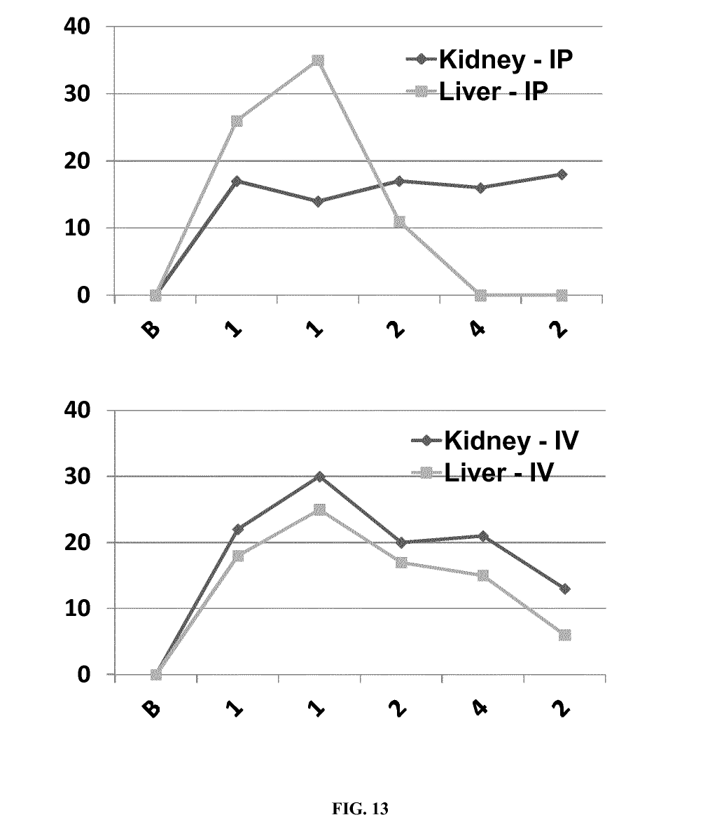

The expression of mRNA-CD40 is displayed in FIG. 13. siRNA-CD40 reduced almost by half the mRNA-CD40 expression after LPS injection after different time intervals. This reduction was mainly observed with LPS injection at 4 hours, one day and 3 days after siRNA-CD40 administration. These data suggest that the time dose intervals of siRNA in experimental models in rodents could range between 3 and 7 days.

1-7. (canceled)

8. Method for the prevention and/or the treatment of a disease in a subject in need thereof, wherein said disease is selected from the group consisting of lupus nephritis, renal ischemia/reperfusion injury and sepsis, and wherein said method comprises the administration to said subject of an interfering RNA that silences CD40 gene expression or of a polynucleotide coding for said interfering RNA.

9. The method according to claim 8, wherein the interfering RNA is targeted to a region in the CD40 mRNA selected from the group consisting of a region located at positions 173-193, 192-212, 479-499, 709-729, 62-82, 137-157, 214-234, 242-262 or 188-214 of the human CD40 mRNA wherein the numbering corresponds to the position with respect to the start codon in the CD40 cDNA as defined in NCBI accession X60592.1.

10. The method according to claim 8, wherein the interfering RNA comprises a sequence as defined in Table 1.

11. The method according to claim 8, wherein the interfering RNA is a short interfering RNA (siRNA).

12. The method according to claim 8, wherein the interfering RNA is administered subcutaneously, intradermally, intramuscularly, intraocularly, intrathecally, intracerebellarly, intranasally, intratracheally, hypodermically, intraperitoneally, intrahepatically, intratesticularly, intratumorally, hypodermically, by injection or by intravascular perfusion.

13. The method according to claim 8, wherein the subject is a mammal.

14. The method according to claim 13, wherein the subject is a human.
